(19) United States
(12) Patent Application Publication
(10) Pub. No.: US 2007/0016101 A1
(43) Pub. Date: Jan. 18, 2007

(54) Core Biopsy Device

(76) Inventors: Dennis D. Feldman, Apollo Beach, FL (US); Heather S. Hanson, San Antonio, TX (US); Mark R. Heistand, San Antonio, TX (US); Theodore J. Kramer, San Antonio, TX (US)

Correspondence Address: SMITH HOPEN, PA, 180 PINE AVENUE NORTH, OLDSMAR, FL, 34677, US

(21) Appl. No.: 11/457,308

(22) Filed: Jul. 13, 2006

Related U.S. Application Data
(60) Provisional application No. 60/595,546, filed on Jul. 13, 2005.

(51) Int. Cl. A61B 10/00

(52) U.S. Cl. 600/567; 600/564

(57) ABSTRACT

A biopsy device used to capture a tissue sample by placing the tissue under tension, thus allowing a greater sized sample with increased quality. The device uses a rotating cannula and stylet with a concave “sample notch.” The rotation of the stylet places the tissue under tension while the rotation of the cannula as it is fired severs the sample.

### CROSS REFERENCE TO RELATED APPLICATIONS

This application claims priority to currently pending U.S. Provisional Patent Application 60/595,546, filed Jul. 13, 2005.

### FIELD OF THE INVENTION

The invention relates generally to biopsy needles. Specifically, the present invention relates to improvements to conventional tissue biopsy devices and results in a larger core sample.

### BACKGROUND OF THE INVENTION

A number of biopsy needles of the prior art have been designed to capture a tissue sample in a stylet having a purchasing recess, into which the sample prolapses after the needle has been inserted. Generally, a cutting cannula is fired thus severing the sample and trapping the sample within the recess.

U.S. Pat. No. 5,718,237 to Haaga describes a biopsy needle that has a coaxial, telescopically interengaged stylet, inner and outer cannulas (the stylet axially and rotatably displaceable relative to the cannula). The stylet has a distal portion provided with a cutting recess (notch) for severing a biopsy specimen, and the inner cannula has a distal portion for cutting and capturing the specimen in the recess. In operation, the device is inserted into the target area and the stylet is displaced into the tissue. The notch is thereby placed within the lesion. The stylet is then rotated about its axis to sever the tissue. The inner cannula is then displaced to cover the stylet and trap the tissue within the lesion. The inner cannula is equipped with a cutting edge to sever tissue as it is displaced.

The '237 patent to Haaga relies on the cutting action of the inner stylet to purchase the lesion, therefore relying on the natural prolapse of tissue into the purchasing recess. Furthermore, the '237 does not provide a rotating cannula which serves to make a cleaner cut and capture a larger sample within the purchasing recess.

U.S. Pat. No. 6,673,023 and U.S. Patent Application 2004/0059254 to Pflueger describe an apparatus for removing tissue and/or other material from a patient. The biopsy device includes a hand piece and a tissue removal mechanism. The tissue removal mechanism includes a cannula having an open distal tip. The mechanism further includes a rotatable element having a distal portion with helical threading. The distal portion of the rotatable element extends beyond the open distal tip of the cannula in order to allow tissue to prolapse between turns of the helical threading. The apparatus is designed to draw soft tissue into the cannula upon rotation of the rotatable element and without the need for supplemental sources of aspiration. The '254 application relies on a helical structure, rather than a sample notch, to draw tissue into the cannula.

Therefore, what is needed is a core biopsy device which captures a biopsy sample under tension greater than that provided by the natural prolapse of the tissue, thus providing a larger sample for analysis.

### SUMMARY OF THE INVENTION

The present invention includes a core biopsy needle having a tubular outer cannula and a telescopically integrated stylet disposed within the outer cannula whereby the stylet and cannula are coaxial in orientation. A purchasing recess is disposed within the stylet whereby the stylet and cannula are rotateable along a common axis in opposite directions such that the counter-rotation of each severs and captures a biopsy sample within the purchasing recess. The rotating stylet initially places the targeted tissue under tension maximizing the quantity of specimen by overfilling/gathering tissue in the sampling notch; secondly the counter rotating cutting cannula takes advantage of said tissue hence cutting a larger more intact/quality sample.

### BRIEF DESCRIPTION OF THE DRAWINGS

FIG. 1 is an isometric view of the present invention without specimen before it is inserted.

FIG. 2 is a cross-sectional view of the sidewall of the receiving recess.

FIG. 3 is an isometric view of one embodiment of the device wherein the receiving recess as a leading side wall with a length greater than the linear length of the recess.

FIG. 4 is an isometric view of the cutting edge of the cannula showing one embodiment wherein the cutting edge is helical.

FIGS. 5-7 are isometric views of the insertion of needle before it is rotated.

FIGS. 8-10 are isometric views of the counter rotation occurring on both outer cannula and inner stylet.

FIGS. 11-13 are isometric views of the needle rotating while it cuts and secures the specimen.

FIGS. 14-16 are isometric views of the needle being withdrawn with sample specimen in tact.

FIG. 17 is an isometric view of needle after it is withdrawn with specimen secured within.

FIG. 18 is an isometric view of needle showing accessibility to specimen.

FIG. 19 is an isometric view of the inventive apparatus detailing the cutting point of one embodiment, where the cutting edge of the cannula meets the leading side wall of the receiving recess.

### DETAILED DESCRIPTION OF THE PREFERRED EMBODIMENTS

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

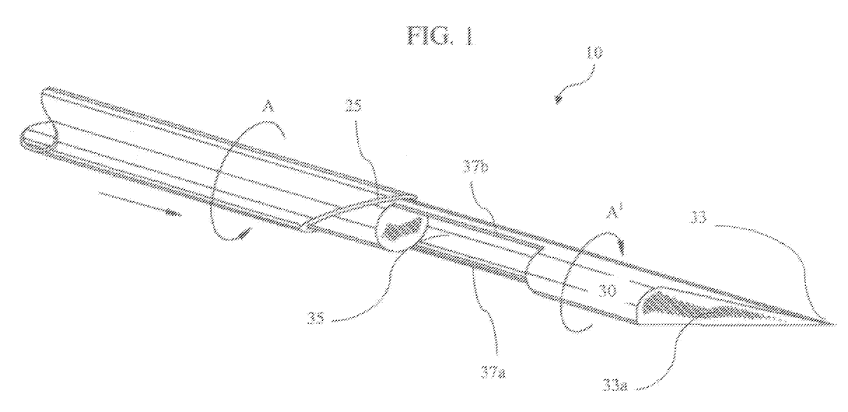

Referring now to FIG. 1, a preferred embodiment of the invention is shown illustrating the component parts of the novel biopsy device 10 in accordance with the present invention. As shown in FIG. 1, biopsy device 10 includes tubular outer cannula 20 which carries solid circular stylet 30. Outer cannula 20 is circular in cross-section, has an axis and circular passageway there through. Outer cannula 20 and stylet 30 are coaxial when assembled and have a common axis. Outer cannula 20 is equipped with distal portion 23 which is preferably tapered. The end of outer cannula 20 proximal to the user is generally equipped with a handle by which biopsy device 10 is manipulated during use. In one embodiment cutting edge 25 of cannula 20 is beveled or otherwise adapted to present a helical cutting surface.

Stylet 30 is solid and circular in cross-section, having a diameter which allows the stylet to be received within cannula 20 and supported therein for axial and rotative displacement relative thereto. Stylet 30 includes distal portion 33 and a proximal portion (not shown) extending axially there from. In a general embodiment, proximal end of cannula 20 and stylet 30 are provided with a handle to facilitate manipulation of the biopsy device, particularly with respect to the orientation of cannula 20 and stylet 30 relative to one another. The axially outer end of distal end 33 of stylet 30 is beveled to provide a distal tip 33a. A portion of stylet 30 is radially and axially cut away at a location spaced axially inward from distal end 33 to provide specimen receiving recess 35. Recess 35 includes side walls 37a and 37b which aid in placing the tissue to be sampled at the biopsy site under tension during use of the device.

As shown in FIGS. 1 through 3, stylet 30, is best described as a solid rod; the distal end 33 of which is equipped with a concavity to form specimen receiving recess 35. Receiving recess 35 defines a fully cylindrical volume, said volume having a cross-sectional area only slightly less than that of stylet 30. Receiving recess 35 is bordered along an extent of its perimeter by side wall 37; further comprising leading side wall 37a and trailing side wall 37b.

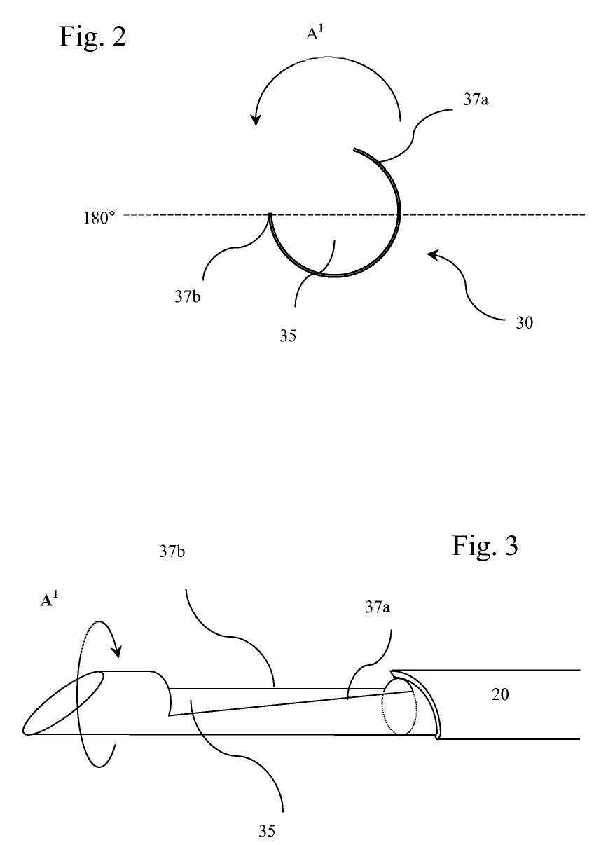

In one embodiment, side wall 37 has a cross-section greater than 180° when viewed along its length (FIG. 2). In addition to enhancing sample collection, discussed below, the extended cross-section of side wall 37 helps to protect large samples from being displaced from recess 35 as cannula 20 is fired over stylet 30. The length of recess 35 can be varied to fit the intended purpose of the device.

In this embodiment, leading side wall 37a has a length greater than the length of recess 35, see FIGS. 2 through 3. In addition to the protective function, discussed above, this leading edge design provides a greater contact surface area between stylet 30 and the sample tissue. Accordingly, leading wall 37a can be adapted with a surface designed to grasp, or aid in the severing of tissue as stylet 30 rotates. FIG. 2 shows cross-section with constant wall thickness for the stylet. In alternate embodiments, however, receiving area 35 has a variable wall thickness with more material in the wall at the central region of the cross-section.

Referring now to FIGS. 1 through 4, cannula 20 is an elongated, tubular member having an enclosed section comprising an annular wall, defining a lumen there through. The inner diameter of the lumen is somewhat greater than the outer diameter of stylet 30, to provide a sliding fit of the stylet therein.

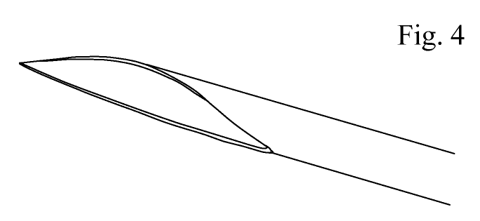

Cannula 20 terminates at its distal end in cutting edge 25. In one embodiment, cutting edge 25 on the distal end of cannula 20 is inclined relative to the longitudinal axis of the cannula to define an elliptical beveled edge. Cutting edge 25 can be provided with a secondary bevel, thereby sharpening the beveled edge and enhancing the severing capability of the cannula. Alternatively, cutting edge 25 of cannula 20 can be manufactured with a variety of shapes including, but not limited to, spherical, conical, cylindrical and helical (see FIG. 4). It is preferable that cutting edge 25 be made of a material which can provide a high degree of sharpness, i.e. steel or ceramics. In another embodiment, all steel components can be made of non-ferrous material metals for use in MRI applications.

When assembled, cannula 20 and stylet 30 are displaceable between retracted and extended positions relative to one another. Prior to use both elements are in the retracted position. FIGS. 2 through 4 illustrate the biopsy device in use, beginning with both cannula 20 and stylet 30 are in the retracted position.

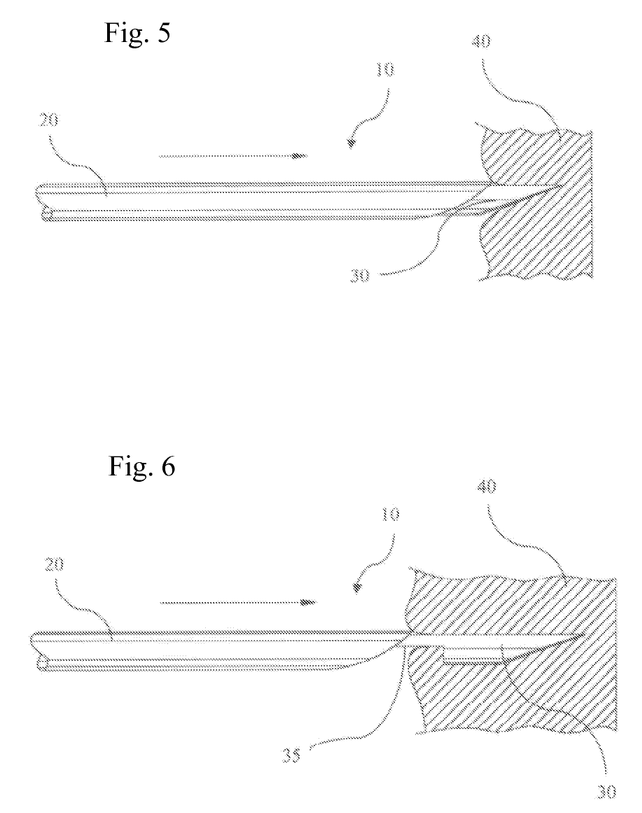

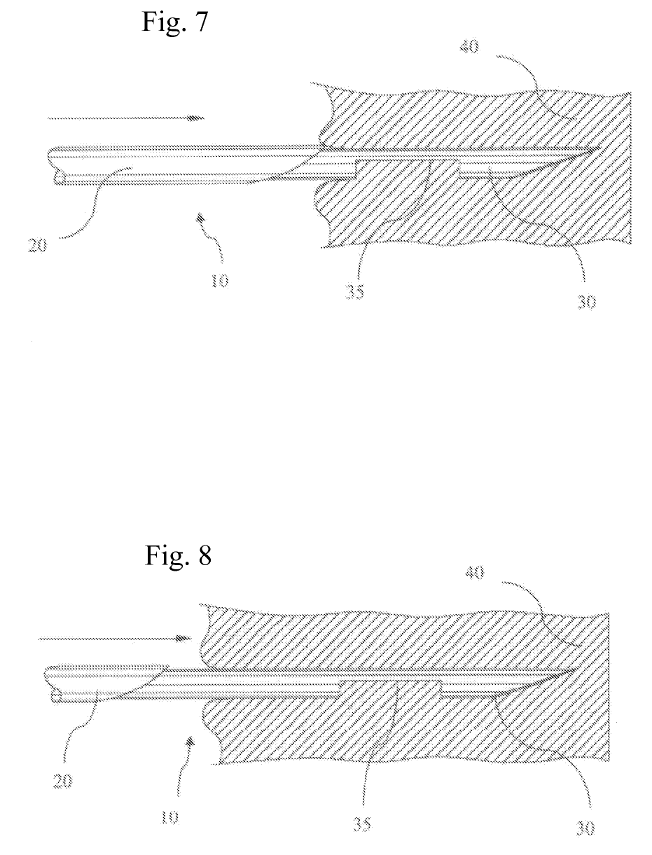

In an embodiment illustrative of the operation of the device, cannula 20 and stylet 30 are urged forward, with the stylet in the retracted position, into the body of a subject to a point adjacent the lesion to be sampled 40. Once cannula 20 and stylet 30 are proximate to lesion 40, distal end 33 of stylet 30 is urged forward and enters lesion 40 in leading relation to cannula 20 (FIGS. 5 through 7). When so positioned, stylet 30 is displaced axially outward relative to cannula 20 from its retracted position to its extended position in which recess 35 is located at the biopsy site as shown in FIG. 8.

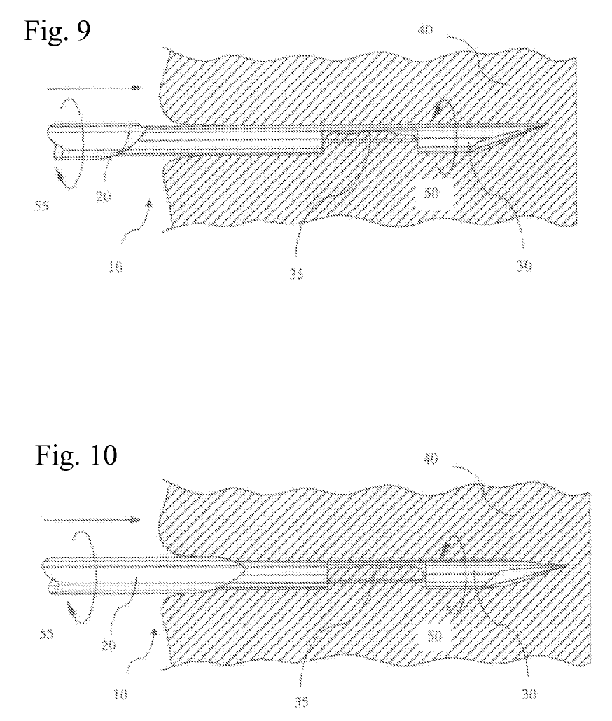

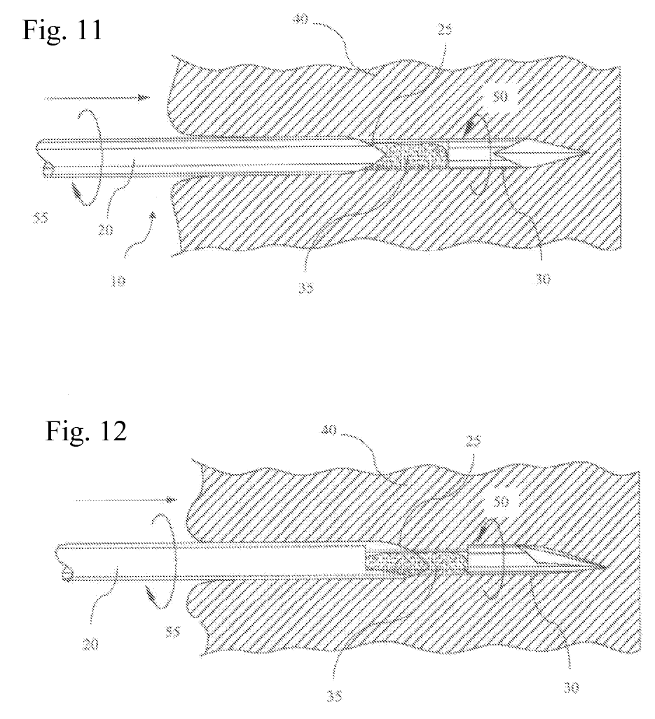

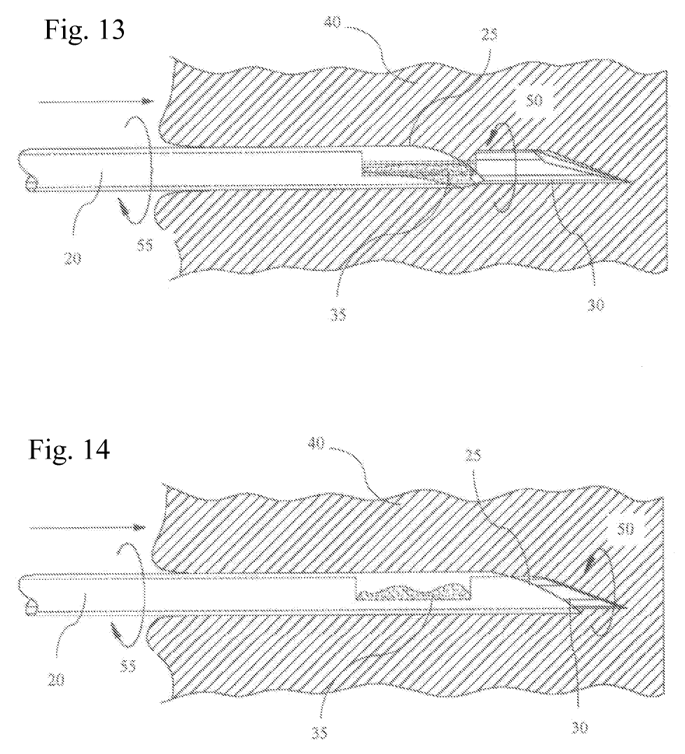

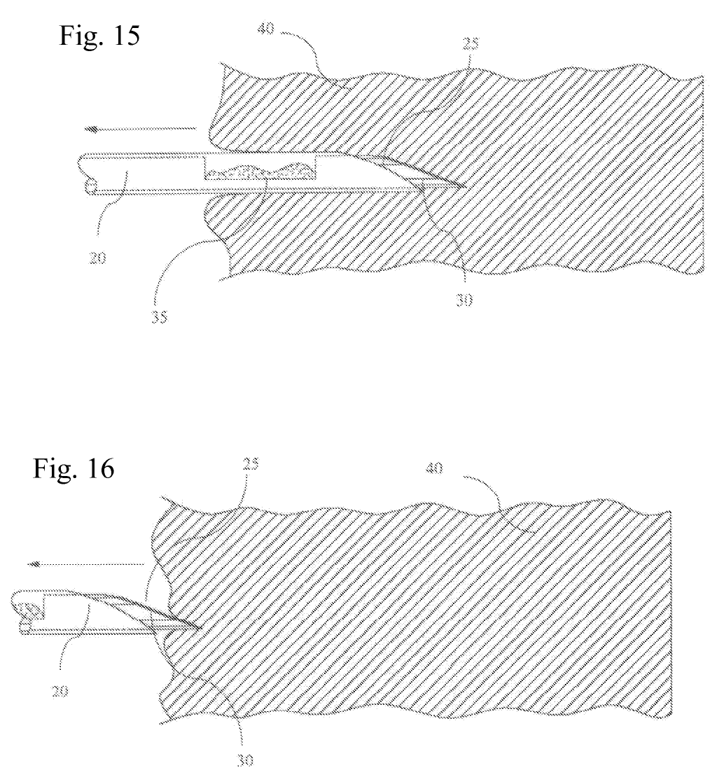

Stylet 30 rotates about its axis as indicated by arrow A1, FIGS. 9 through 14, urging the tissue specimen at the biopsy site into recess 35. Cannula 20 is then displaced axially outwardly relative its retracted position. Cannula 20 is rotated about its axis in the opposite direction of stylet 30 as indicated by arrow A in FIGS. 9-14. In one embodiment, during the movement of cannula 20, cutting edge 25 at the distal end thereof severs the tissue at the biopsy site into recess 35 of stylet 30. In passing axially and radially across recess 35, cannula 20 radially captures a larger biopsy specimen therein (FIG. 15).

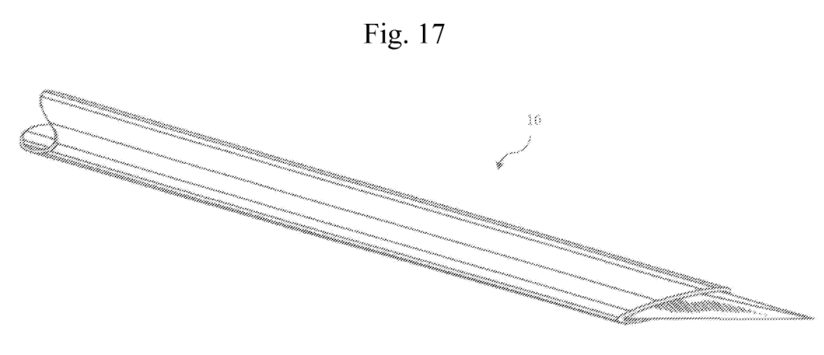

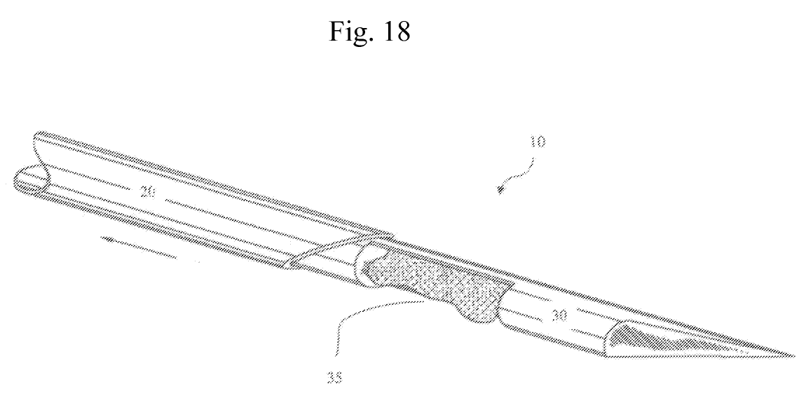

Once the specimen is captured within recess 35 of stylet 30, the biopsy device is withdrawn (FIGS. 15 through 17). Once fully withdrawn, as shown in FIG. 18, cannula 20 is retracted exposing recess 35 of stylet 30 which now houses biopsy specimen 40a.

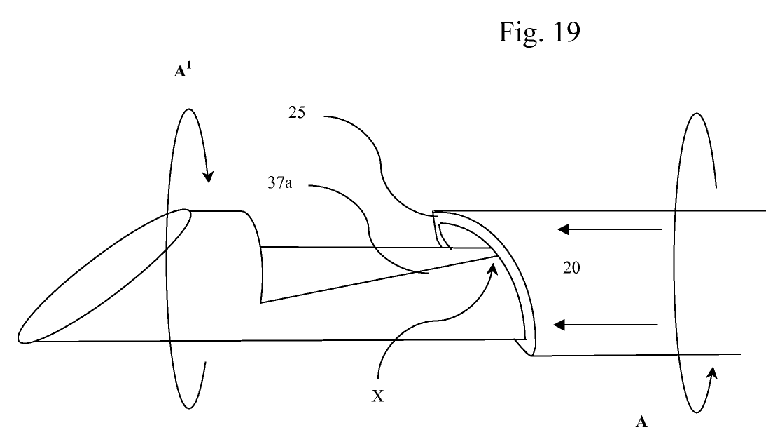

Stylet 30 and cannula 20 work in conjunction to sever and trap the sample in recess 35. The rotation of both stylet 30 and cannula 20 is such that the sample is severed at cutting point X (FIG. 19). As detailed in FIG. 19, cutting edge 25 of cannula 20 and leading wall 37a of stylet 30 meet at cutting point X during their rotation. When cannula 30 is fired to the severing position, the sample is severed from the surrounding tissue by the combination of the force exerted by the rotation of stylet 30 and the movement (linear and/or rotating) of cannula 20.

The rotation of stylet 30 and cannula 20 results in the device severing the sample from the surrounding tissue; thereby forming a substantially cylindrical sample. In one embodiment, cannula 20 is rotated approximately 1½ turns, relative to stylet 30, to ensure that the sample has been completely severed from the surrounding tissue.

In an alternate embodiment, rotation of the cannula is limited to prevent the leading point of cannula 20 from entering into the cutting area of the tissue during rotation. The angle between cutting edge 25 of cannula 20 and leading wall 37a of stylet 30 are roughly inverted in this embodiment as the cutting edge 25 of cannula 20 passes over leading wall 37a of stylet 30 (see FIG. 19).

In another embodiment, cutting edge 25 of cannula 20 and leading wall 37a of stylet 30 form an acute angle at cutting point X such that the cutting action is like that of a scissor at point X; however, at locations away from point X, the cutting action is similar to a standard biopsy device. Cutting edge 25 of cannula 20 may alternatively be angled such that it forms an obtuse angle with leading wall 37a of stylet 30. In this embodiment, the motion shown in FIG. 19 results in somewhat of a slicing action; like that of a knife. While these incident angles are illustrative of the inventive device, they are not intended to be limiting.

However, the rotation need only be sufficient to ensure the separation of the sample from the tissue mass. Rotation can therefore encompass multiple or even partial revolutions of the cannula and/or stylet; as well as combinations thereof. This severing action remains effective in embodiments wherein the stylet and cannula rotate the same direction or in counter rotation; as well as in embodiments wherein the stylet and cannula rotate at different times during the taking of the sample.

In some embodiments, the inventive device can include a firing mechanism that includes a first and second trigger configured to selectively control the operation of the stylet and the cannula. The firing mechanism can also be adapted to actuate both the cannula and stylet sequentially. For example, the advancement of cannula 20 to the severing position can be done as part of, or separate from, the rotation of stylet 30. In one embodiment, the advancement of cannula 20 is accomplished in the same step as the rotation of stylet 30.

While the cannula and stylet of the present invention are described herein as being provided with handles for manipulation of the component parts relative to one another, it will be appreciated that the biopsy device is operable in the manner of a standard side cut needle whereby these component parts are adaptable to automated operation. These and other modifications of the preferred embodiment, as well as other embodiments of the present invention, will be obvious to those skilled in the art from the disclosure of the preferred embodiment herein.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A biopsy device for obtaining a sample, comprising:
a tubular outer cannula having a cutting edge; and
a stylet having a recess disposed near a distal end thereof;
said cannula being axially extendable over said stylet;
said stylet being slidably and rotatably disposed within said cannula.

2. The biopsy device of claim 1, further comprising a firing mechanism adapted to rotate said stylet.

3. The biopsy device of claim 1 wherein the firing mechanism is adapted to rotate the cannula.

4. The biopsy device of claim 3 wherein the cannula rotates in the opposite direction of the stylet.

5. The biopsy device of claim 3 wherein the cannula rotates in the same direction as the stylet.

6. The biopsy device of claim 1 wherein the cutting edge of the cannula is substantially straight.

7. The biopsy device of claim 1 wherein the cutting edge of the cannula is substantially helical.

8. The biopsy device of claim 1 wherein the extension of the cannula is triggered concurrently with the rotation of the stylet.

9. The biopsy device of claim 1 wherein the extension of the cannula is triggered subsequent to the rotation of the stylet.

10. The biopsy device of claim 3 wherein the rotation of the cannula is triggered concurrently with the rotation of the stylet.

11. The biopsy device of claim 3 wherein the rotation of the cannula is triggered subsequent to the rotation of the stylet.

12. The biopsy device of claim 3 wherein the recess further comprises a side wall forming a leading edge with regard to the stylet's rotation.

13. The biopsy device of claim 12 wherein the leading edge of the recess is greater than the length of the recess.

14. The biopsy device of claim 13 wherein the leading edge defines sample protecting compartment.

15. The biopsy device of claim 12 wherein the sidewall has a variable wall thickness.

16. The biopsy device of claim 15 wherein the side wall is wider at the central region of stylet's cross-section.

17. The biopsy device of claim 12 wherein the angle between the cutting edge of the cannula and the leading wall of the stylet are substantially inverted as the cutting edge of the cannula passes over the leading wall of the stylet.

18. The biopsy device of claim 12 wherein the cutting edge of the cannula and leading wall of the stylet form an acute angle as the cutting edge of the cannula passes over the leading wall of the stylet to form a cutting point.

19. The biopsy device of claim 12 wherein the cutting edge of the cannula and leading wall of the stylet form an obtuse angle as the cutting edge of the cannula passes over the leading wall of the stylet to form a cutting point.

20. The biopsy device of claim 1 wherein the firing mechanism rotates said stylet prior to axially extending the cannula over said stylet.

21. The biopsy device of claim 1 wherein the stylet rotates for at least a portion of its forward motion.

22. The biopsy device of claim 3 wherein the cannula rotates for at least a portion of its forward motion.

23. A method of obtaining a biological sample, comprising the steps of:
introducing a stylet having a recess disposed near the distal end thereof, proximate to the sample;
rotating said stylet; and
axially extending a tubular cannula, said cannula having a cutting edge, over said stylet.

24. The method of claim 23 further comprising the step of rotating the cannula.

25. The method of claim 24 wherein the cannula rotates in the opposite direction of the stylet.

26. The method of claim 24 wherein the cannula rotates in the same direction as the stylet.

27. The method of claim 23 wherein the cutting edge of the cannula is substantially straight.

28. The method of claim 23 wherein the cutting edge of the cannula is substantially helical.

29. The method of claim 23 wherein the extension of the cannula is actuated concurrently with the rotation of the stylet.

30. The method of claim 23 wherein the extension of the cannula is actuated subsequent to the rotation of the stylet.

31. The method of claim 24 wherein the rotation of the cannula is actuated concurrently with the rotation of the stylet.

32. The method of claim 24 wherein the rotation of the cannula is actuated subsequent to the rotation of the stylet.

33. The method of claim 24 wherein the recess is adapted with a side wall forming a leading edge with regard to the stylet's rotation.

34. The method of claim 33 wherein the leading edge of the recess is greater than the length of the recess.

35. The method of claim 33 wherein the leading edge defines sample protecting compartment.

36. The method of claim 33 wherein the sidewall has a variable wall thickness.

37. The method of claim 36 wherein the side wall is wider at the central region of stylet's cross-section.

38. The method of claim 33 wherein the angle between the cutting edge of the cannula and the leading wall of the stylet are substantially inverted as the cutting edge of the cannula passes over the leading wall of the stylet.

39. The method of claim 33 wherein the cutting edge of the cannula and leading wall of the stylet form an acute angle as the cutting edge of the cannula passes over the leading wall of the stylet to form a cutting point.

40. The method of claim 33 wherein the cutting edge of the cannula and leading wall of the stylet form an obtuse angle as the cutting edge of the cannula passes over the leading wall of the stylet to form a cutting point.

41. The method of claim 23 wherein the stylet is rotated prior to axially extending the cannula over said stylet.

42. The method of claim 23 wherein the stylet is rotated for at least a portion of its forward motion.

43. The method of claim 24 wherein the cannula is rotated for at least a portion of its forward motion.
